(19) United States
(12) Patent Application Publication
(10) Pub. No.: US 2021/0038650 A1
(43) Pub. Date: Feb. 11, 2021

(54) MESENCHYMAL STEM CELL THERAPY OF LEIGH SYNDROME

(71) Applicant: CELL MEDICINE, INC., Farmers Branch, TX (US)

(72) Inventor: Neil RIORDAN, Westlake, TX (US)

(73) Assignee: CELL MEDICINE, INC., Farmers Branch, TX (US)

(21) Appl. No.: 16/758,520

(22) PCT Filed: Oct. 23, 2018
(86) PCT No.: PCT/US2018/057091

§ 371 Date: Apr. 23, 2020

Related U.S. Application Data
(60) Provisional application No. 62/576,025, filed on Oct. 23, 2017.

(51) Int. Cl. A61K 35/28; A61P 25/00; C12N 5/0775; A61K 35/51

(52) U.S. Cl. CPC A61K 35/28; A61P 25/00; A61K 9/0043; A61K 35/51; C12N 5/0665

(57) ABSTRACT

Disclosed are means, methods and treatments of Leigh Syndrome, using mesenchymal stem cells. In one particular embodiment, mesenchymal stem cells are administered for the purposes of reducing disease progression, and reversing disease. Said mesenchymal stem cells may be generated according to the invention, by selection of markers specifically upregulated or downregulated on enhanced cells as compared to majority of mesenchymal stem cells. The invention further provides means of co-administration of mesenchymal stem cells with lysates, conditioned media, or exosomes of said mesenchymal stem cells to enhance therapeutic activity.

### CROSS-REFERENCE TO RELATED APPLICATIONS

This invention claims benefit of priority to U.S. Provisional Application No. 62/576,025, filed Oct. 23, 2017, the contents of which are incorporated herein by reference.

### FIELD OF THE INVENTION

The invention pertains to the field of treatment of Leigh Syndrome, more specifically, the invention pertains to the use of stem cells for treatment of gene therapy, more specifically, the invention provides stem cell therapies and protocols for inhibiting and/or reversing Leigh Syndrome.

### BACKGROUND

Leigh syndrome (or subacute necrotizing encephalomyelopathy) is characterized by onset of symptoms typically between ages three and 12 months, often following a viral infection. Decompensation (often with elevated lactate levels in blood and/or CSF) during an intercurrent illness is typically associated with psychomotor retardation or regression. Neurologic features include hypotonia, spasticity, movement disorders (including chorea), cerebellar ataxia, and peripheral neuropathy. Extraneurologic manifestations may include hypertrophic cardiomyopathy. About 50% of affected individuals die by age three years, most often as a result of respiratory or cardiac failure [1]. Criteria for diagnosis of Leigh syndrome are as follows: (1) a neurodegenerative disease with variable symptoms, (2) caused by mitochondrial dysfunction from a hereditary genetic defect and (3) accompanied by bilateral central nervous system lesions. A genetic etiology is confirmed in approximately 50% of patients, with more than 60 identified mutations in the nuclear and mitochondrial genomes. A typical study detailing clinical features and imaging studies described a cohort of 17 children with genetically confirmed Leigh syndrome. MR findings include lesions in the brainstem in 9 children (53%), basal ganglia in 13 (76%), thalami in 4 (24%) and dentate nuclei in 2 (12%), and global atrophy in 2 (12%). The brainstem lesions were most frequent in the midbrain and medulla oblongata. With follow-up an increased number of lesions from baseline was observed in 7 of 13 children, evolution of the initial lesion was seen in 6, and complete regression of the lesions was seen in 3. No cerebral white matter lesions were found in any of the 17 children. This representative sample that was published is In concordance with the standard description of Leigh syndrome in the literature, where it is described to follow pattern of bilateral, symmetrical basal ganglia or brainstem changes. Lesions in Leigh syndrome evolve over time and a lack of visible lesions does not exclude the diagnosis. Reversibility of lesions is seen in some patients, making the continued search for treatment and prevention a priority for clinicians and researchers [2].

In the diagnosis of Leigh syndrome, it is recommended that both biochemical and genetic markers, due to discrepancy in some cases between the two. For example, in one study, the clinical validity of various diagnostic tools in confirming mitochondrial respiratory complex (MRC) disorder in Leigh syndrome (LS) and Leigh-like syndrome (LL) was assessed. The results of enzyme assays, molecular analysis, and cellular oxygen consumption rate (OCR) measurements were examined. Of 106 patients, 41 were biochemically and genetically verified, and 34 had reduced MRC activity but no causative mutations. Seven patients with normal MRC complex activities had mutations in the MT-ATP6 gene. Five further patients with normal activity in MRC were identified with causative mutations. Conversely, 12 out of 60 enzyme assays performed for genetically verified patients returned normal results. No biochemical or genetic background was confirmed for 19 patients. OCR was reduced in ten out of 19 patients with negative enzyme assay results. Inconsistent enzyme assay results between fibroblast and skeletal muscle biopsy samples were observed in 33% of 37 simultaneously analyzed cases. These data suggest that highest diagnostic rate is reached using a combined enzymatic and genetic approach, analyzing more than one type of biological materials where suitable. Microscale oxygraphy detected MRC impairment in 50% cases with no defect in MRC complex activities [3].

Scientific study of Leigh syndrome can be performed in animal models. For example, Ndufs4 knockout (Ndufs4(−/−)) mouse, is model of mitochondrial complex I deficiency. Ndusf4(−/−) mice exhibit progressive neurodegeneration, which closely resemble the human Leigh syndrome phenotype. When dissecting behavioral abnormalities in animal models it is of great importance to apply translational tools that are clinically relevant. To distinguish gait abnormalities in patients, simple walking tests can be assessed, but in animals this is not easy. In one study, the automated CatWalk gait analysis tool was used in the Ndufs4(−/−) mouse model. Marked differences were noted between Ndufs4(−/−) and control mice in dynamic, static, coordination and support parameters. Variation of walking speed was significantly increased in Ndufs4(−/−) mice, suggesting hampered and uncoordinated gait. Furthermore, decreased regularity index, increased base of support and changes in support were noted in the Ndufs4(−/−) mice. Here, we report the ability of the CatWalk system to sensitively assess gait abnormalities in Ndufs4(−/−) mice. This objective gait analysis can be of great value for intervention and drug efficacy studies in animal models for mitochondrial disease [4].

In another study targeting exon 2 of Ndufs4 to delete the NDUFS4 protein in mouse embryos to mimic Leigh syndrome was performed. Then, then the described the phenotypes of our mouse model by forced swimming and the open-field test as well as by assessing other behavioral characteristics. Intracytoplasmic sperm injection (ICSI) was performed to obtain KO embryos to test the influence of NDUFS4 deletion on early embryonic development. In this study, they first generated Ndufs4 KO mice with physical and behavioral phenotypes similar to Leigh syndrome using the CRISPR/Cas9 system. The low developmental rate of KO embryos that were derived from knockout gametes indicated that the absence of NDUFS4 impaired the development of preimplantation embryos [5].

At a functional level, NADH dehydrogenase (ubiquinone) Fe—S protein is encoded by Ndufs4, a nuclear gene that transcribes an 18 kDa protein that is one of 46 subunits of the mitochondrial complex I; it is required for the complete assembly and function of complex I. The Ndufs4 knockout (NKO) mouse is a model of human Leigh Syndrome, exhibiting similar symptomology to the human condition including ataxic, encephalomyopathy, lethargy, loss of motor skill, blindness, and elevated serum lactate [6-8].

Mouse models or human patients deficient in Ndufs4 possess reduced Complex I levels and activity, and mutations in Ndufs4 cause Leigh Syndrome in humans [9]. NKO mice are small but develop normally until about postnatal day 35 (P35) when they begin to display characteristic neurological phenotypes, progressive neuroinflammation and neurodegeneration, and brain lesions similar to those present in human Leigh Syndrome patients. NKO mice also show a profound decrease of body fat compared to their wild type (WT) or heterozygous littermates, and typically die between P50 and P60 [10].

S6K1 is a ribosomal protein that when disrupted in the Ndufs4 knockout mouse model of Leigh Syndrome results in prolonged survival. Interestingly, disruption of S6K1 in the liver only was sufficient to prolong survival of the Ndufs4 knockout mice [11].

To gain insight into the systemic, biochemical consequences of respiratory chain dysfunction, a studed was performed in a case-control, prospective metabolic profiling study in a genetically homogenous cohort of patients with Leigh syndrome French Canadian variant, a mitochondrial respiratory chain disease due to loss-of-function mutations in LRPPRC. Forty-five plasma and urinary analytes discriminating patients from controls, including classic markers of mitochondrial metabolic dysfunction (lactate and acylcarnitines), as well as unexpected markers of cardiometabolic risk (insulin and adiponectin), amino acid catabolism linked to NADH status (α-hydroxybutyrate), and NAD(+) biosynthesis (kynurenine and 3-hydroxyanthranilic acid) where found. These studies identify systemic, metabolic pathway derangements that can lie downstream of primary mitochondrial lesions, with implications for understanding how the organelle contributes to rare and common diseases [12].

It is known that mitochondria are key regulators of cellular homeostasis, and mitochondrial dysfunction is strongly linked to neurodegenerative diseases, including Alzheimer's and Parkinson's. Mitochondria communicate their bioenergetic status to the cell via mitochondrial retrograde signaling. To investigate the role of mitochondrial retrograde signaling in neurons, one study induced mitochondrial dysfunction in the Drosophila nervous system. Neuronal mitochondrial dysfunction causes reduced viability, defects in neuronal function, decreased redox potential, and reduced numbers of presynaptic mitochondria and active zones. The investigators found that neuronal mitochondrial dysfunction stimulates a retrograde signaling response that controls the expression of several hundred nuclear genes. It was shown that the Drosophila hypoxia inducible factor alpha (HIFa) ortholog Similar (Sima) regulates the expression of several of these retrograde genes, suggesting that Sima mediates mitochondrial retrograde signaling. Remarkably, knockdown of Sima restores neuronal function without affecting the primary mitochondrial defect, demonstrating that mitochondrial retrograde signaling is partly responsible for neuronal dysfunction. Sima knockdown also restores function in a Drosophila model of the mitochondrial disease Leigh syndrome and in a Drosophila model of familial Parkinson's disease. Thus, mitochondrial retrograde signaling regulates neuronal activity and can be manipulated to enhance neuronal function, despite mitochondrial impairment [13].

Elevated fumarate concentrations as a result of Krebs cycle inhibition lead to increases in protein succination, an irreversible post-translational modification that occurs when fumarate reacts with cysteine residues to generate S-(2-succino)cysteine (2SC) [14]. Metabolic events that reduce NADH re-oxidation can block Krebs cycle activity [15]; therefore it was hypothesized that oxidative phosphorylation deficiencies, such as those observed in some mitochondrial diseases, would also lead to increased protein succination. Using the Ndufs4 knockout (Ndufs4 KO) mouse, a model of Leigh syndrome, it was demonstrated for the first time that protein succination is increased in the brainstem (BS), particularly in the vestibular nucleus. Importantly, the brainstem is the most affected region exhibiting neurodegeneration and astrocyte and microglial proliferation, and these mice typically die of respiratory failure attributed to vestibular nucleus pathology. In contrast, no increases in protein succination were observed in the skeletal muscle, corresponding with the lack of muscle pathology observed in this model. 2D SDS-PAGE followed by immunoblotting for succinated proteins and MS/MS analysis of BS proteins allowed us to identify the voltage-dependent anion channels 1 and 2 as specific targets of succination in the Ndufs4 knockout. Using targeted mass spectrometry, Cys(77) and Cys(48) were identified as endogenous sites of succination in voltage-dependent anion channels 2. Given the important role of voltage-dependent anion channels isoforms in the exchange of ADP/ATP between the cytosol and the mitochondria, and the already decreased capacity for ATP synthesis in the Ndufs4 KO mice, it was proposed that the increased protein succination observed in the BS of these animals would further decrease the already compromised mitochondrial function. These data suggest that fumarate is a novel biochemical link that may contribute to the progression of the neuropathology in this mitochondrial disease model [16].

Basal ganglia nuclei, including the striatum, are affected in LS patients. However, neither the identity of the affected cell types in the striatum nor their contribution to the disease has been established. Here, a mouse model of LS lacking Ndufs4, a mitochondrial complex I subunit, was used to confirm that loss of complex I, but not complex II, alters respiration in the striatum. To assess the role of striatal dysfunction in the pathology, the investigators selectively inactivated Ndufs4 in the striatal medium spiny neurons (MSNs), which account for over 95% of striatal neurons. The results showed that lack of Ndufs4 in MSNs causes a non-fatal progressive motor impairment without affecting the cognitive function of mice. Furthermore, no inflammatory responses or neuronal loss was observed up to 6 months of age. Hence, complex I deficiency in MSNs contributes to the motor deficits observed in LS, but not to the neural degeneration, suggesting that other neuronal populations drive the plethora of clinical signs in LS [17].

Although there is increasing knowledge of the biology surrounding LS, no curative treatments exist. Some therapeutics that have been attempted include, KH176, a new chemical entity derivative of Trolox, which was assessed in in Ndufs4^{-/-} mice. Using in vivo brain diffusion tensor imaging, it was shown that there occurs a loss of brain microstructural coherence in Ndufs4^{-/-} mice in the cerebral cortex, external capsule and cerebral peduncle. These findings are in line with the white matter diffusivity changes described in mitochondrial disease patients. Long-term KH176 treatment retained brain microstructural coherence in the external capsule in Ndufs4^{-/-} mice and normalized the increased lipid peroxidation in this area and the cerebral cortex. Furthermore, KH176 treatment was able to significantly improve rotarod and gait performance and reduced the degeneration of retinal ganglion cells in Ndufs4^{-/-} mice. Unfortunately, clinical trials have not commenced and there is no means to predict possibility utility in humans given the early stage of this approach in clinical development [18]

Another approach, although somewhat not practical involves manipulation of oxygen levels. It was found that normoxia-treated KO mice die from neurodegeneration at about 60 d, hypoxia-treated mice eventually die at about 270 d, likely from cardiac disease, and hyperoxia-treated mice die within days from acute pulmonary edema. Additionally, it was found that more conservative hypoxia regimens, such as continuous normobaric 17% O₂ or intermittent hypoxia, are ineffective in preventing neuropathology. Finally, the investigators showed that breathing normobaric 11% O₂ in mice with late-stage encephalopathy reverses their established neurological disease, evidenced by improved behavior, circulating disease biomarkers, and survival rates. Importantly, the pathognomonic MRI brain lesions and neurohistopathologic findings are reversed after 4 wk of hypoxia. Upon return to normoxia, Ndufs4 KO mice die within days [19]. The therapeutic benefit of hypoxia was demonstrated in another animal model paper [20].

Another group evaluated effects of TOR inhibition in a Drosophila model of complex I deficiency. Treatment with rapamycin robustly suppresses the lifespan defect in this model of LS, without affecting behavioral phenotypes. Interestingly, this increased lifespan in response to TOR inhibition occurs in an autophagy-independent manner. Further, the investigators identified a fat storage defect in the ND2 mutant flies that is rescued by rapamycin, supporting a model that rapamycin exerts its effects on mitochondrial disease in these animals by altering metabolism [21].

Another approach involved administration of ketogenic diet based on decanoic acid (C10), a component of the medium chain triglyceride KD, and a ligand for the nuclear receptor PPAR-γ known to be involved in mitochondrial biogenesis. The effects of C10 were investigated in primary fibroblasts from a cohort of patients with Leigh syndrome (LS) caused by nuclear-encoded defects of respiratory chain complex I, using mitochondrial respiratory chain enzyme assays, gene expression microarray, qPCR and flow cytometry. Treatment with C10 increased citrate synthase activity, a marker of cellular mitochondrial content, in 50% of fibroblasts obtained from individuals diagnosed with LS in a PPAR-γ-mediated manner. Gene expression analysis and qPCR studies suggested that treating cells with C10 supports fatty acid metabolism, through increasing ACADVL and CPT1 expression, whilst downregulating genes involved in glucose metabolism (PDK3, PDK4). PCK2, involved in blocking glucose metabolism, was upregulated, as was CAT, encoding catalase. Moreover, treatment with C10 also decreased oxidative stress in complex I deficient (rotenone treated) cells. However, since not all cells from subjects with LS appeared to respond to C10, prior cellular testing in vitro could be employed as a means for selecting individuals for subsequent clinical studies involving C10 preparations [22].

### SUMMARY

Certain embodiments are directed to methods of treating a patient suffering from Leigh Syndrome comprising the steps of: a) selecting a patient suffering from Leigh Syndrome in need of treatment; and b) administering to said patient stem cells, and/or products derived from said stem cells at a frequency and concentration sufficient to induce a therapeutic response in said patient.

Certain embodiments are directed to methods of treatment wherein said Leigh Syndrome is subacute necrotizing encephalomyelopathy.

Certain embodiments are directed to methods of treatment wherein said Leigh Syndrome is a condition selected from a group comprising of a) adult-onset subacute necrotizing encephalomyelopathy; b) infantile necrotizing encephalopathy; and c) X-linked infantile nectrotizing encephalopathy

Certain embodiments are directed to methods of treatment wherein said Leigh Syndrome is associated with bilateral lesions characteristic of cellular damage and/or death in the midbrain and brainstem.

Certain embodiments are directed to methods of treatment wherein said Leigh Syndrome is associated with a pyruvate dehydrogenase (PDHC) deficiency.

Certain embodiments are directed to methods of treatment wherein said Leigh Syndrome is associated with a respiratory chain enzyme defect.

Certain embodiments are directed to methods of treatment wherein said respiratory chain defect is a defect in one or more mitochondrial Complexes selected from a group comprising of: a) Complex I; b) Complex II; c) Complex IV and d) Complex V.

Certain embodiments are directed to methods of treatment wherein said Leigh Syndrome is associated with demyelination.

Certain embodiments are directed to methods of treatment wherein said Leigh Syndrome is associated with a mutation in one or more genes selected from a group comprising of: a) AIFM1; b) BCS1L; c) BTD; d) C12orf65; e) COX10; f) COX15; g) DLAT; h) DLD; i) EARS2; j) ECHS1; k) ETHE1; 1) FARS2; m) FBXL4; n) FOXRED1; o) GFM1; p) GFM2; q) GTPBP3; r) HIBCH; s) IARS2; t) LIAS; u) LIPT1; v) LRPPRC; w) MT-ATP6; x) MT-CO3; y) MT-ND1; z) MT-ND2; aa) MT-ND3; ab) MT-ND4; ac) MT-ND4; ad) MT-ND5; ae) MG-ND6; af) MT-TI; ag) MT-TK; ah) MT-TL1; ai) MT-TV; aj) MT-TW; ak) MTFMT; al) NARS2; am) NDUFA1; an) NDUFA2; ao) NDUFA4; ap) NDUFA9; aq) NDUFA10; ar) NDUFA11; as) NDUFA12; at) NDUFAF2; au) NDUFAF5; aw) NDUFAF6; ax) NDUFS1; ay) NDUFS2; az) NDUFS3; ba) NDUFS4; bb) NDUFS7; bc) NDUFS8; bd) NDUFV1; be) NDUFV2; bf) PDHA1; bg) PDHB; bh) PDHX; bi) PDSS2; bj) PET100; bk) PNPT1; bl) POLG; bm) SCO2; bn) SDHA; bo) SDHAF1; bp) SERAC1; bq) SLC19A3; br) SLC25A19; bs) SUCLA2; bt) SUCLG1; bu) SURF1; by) TACO1; bw) TPK1; bx) TRMU; by) TSFM; bz) TTC19; and ca) UQCRQ.

Certain embodiments are directed to methods of treatment wherein administration of stem cells, stem cell derived products, or a mixture thereof, is performed by a means selected from a group of means comprising of: a) intravenous; b) intralymphatic; c) intraperitoneal; d) intrathecal; e) intraventricular; f) intra-arterial; and g) subcutaneous.

Certain embodiments are directed to methods of treatment wherein said stem cells are pluripotent stem cells.

Certain embodiments are directed to methods of treatment wherein said pluripotent stem cells are selected from a group comprising of: a) embryonic stem cells; b) parthenogenic derived stem cells; c) inducible pluripotent stem cells; d) somatic cell nuclear transfer derived stem cells; e) cytoplasmic transfer derived stem cells; and f) stimulus-triggered acquisition of pluripotency.

Certain embodiments are directed to methods of treatment wherein said stem cells are hematopoietic stem cell.

Certain embodiments are directed to methods of treatment wherein said hematopoietic stem cells are capable of multi-lineage reconstitution in an immunodeficient host.

Certain embodiments are directed to methods of treatment wherein said hematopoietic stem cells express the c-kit protein.

Certain embodiments are directed to methods of treatment wherein said hematopoietic stem cells express the Sca-1 protein.

Certain embodiments are directed to methods of treatment wherein said hematopoietic stem cells express CD34.

Certain embodiments are directed to methods of treatment wherein said hematopoietic stem cells express CD133.

Certain embodiments are directed to methods of treatment wherein said hematopoietic stem cells lack expression of lineage markers.

Certain embodiments are directed to methods of treatment wherein said hematopoietic stem cells lack expression of CD38.

Certain embodiments are directed to methods of treatment wherein said hematopoietic stem cells are positive for expression of c-kit and Sca-1 and substantially lack expression of lineage markers.

Certain embodiments are directed to methods of treatment wherein said hematopoietic stem cells are derived from a group of sources, said group comprising of: a) peripheral blood; b) mobilized peripheral blood; c) bone marrow; d) cord blood; e) adipose stromal vascular fraction; and f) derived from progenitor cells.

Certain embodiments are directed to methods of treatment wherein said progenitor cell is a pluripotent stem cell.

Certain embodiments are directed to methods of treatment wherein said stem cells are mesenchymal stem cells.

Certain embodiments are directed to methods of treatment wherein said mesenchymal stem cells are plastic adherent.

Certain embodiments are directed to methods of treatment wherein said mesenchymal stem cells express a marker selected from a group comprising of: a) CD73; b) CD90; and c) CD105.

Certain embodiments are directed to methods of treatment wherein said mesenchymal stem cells lack expression of a marker selected from a group comprising of: a) CD14; b) CD45; and c) CD34.

Certain embodiments are directed to methods of treatment wherein said mesenchymal stem cells are derived from tissues selected from a group comprising of: a) bone marrow; b) peripheral blood; c) adipose tissue; d) mobilized peripheral blood; e) umbilical cord blood; f) Wharton's jelly; g) umbilical cord tissue; h) skeletal muscle tissue; i) subepithelial umbilical cord; j) endometrial tissue; k) menstrual blood; and l) fallopian tube tissue.

Certain embodiments are directed to methods of treatment wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Certain embodiments are directed to methods of treatment wherein said mesenchymal stem cells from umbilical cord tissue do not express markers selected from a group comprising of: a) CD117; b) CD31; c) CD34; and CD45;

Certain embodiments are directed to methods of treatment wherein said mesenchymal stem cells from umbilical cord tissue express, relative to a human fibroblast, increased levels of interleukin 8 and reticulon 1

Certain embodiments are directed to methods of treatment wherein said mesenchymal stem cells from umbilical cord tissue have the potential to differentiate into cells of at least a skeletal muscle, vascular smooth muscle, pericyte or vascular endothelium phenotype.

Certain embodiments are directed to methods of treatment wherein said mesenchymal stem cells from umbilical cord tissue express markers selected from a group comprising of: a) CD10; b) CD13; c) CD44; d) CD73; and e) CD90.

Certain embodiments are directed to methods of treatment wherein said umbilical cord tissue mesenchymal stem cell is an isolated umbilical cord tissue cell isolated from umbilical cord tissue substantially free of blood that is capable of self-renewal and expansion in culture,

Certain embodiments are directed to methods of treatment wherein said umbilical cord tissue mesenchymal stem cells has the potential to differentiate into cells of other phenotypes.

Certain embodiments are directed to methods of treatment wherein said other phenotypes comprise: a) osteocytic; b) adipogenic; and c) chondrogenic differentiation.

Certain embodiments are directed to methods of treatment wherein said cord tissue derived mesenchymal stem cells can undergo at least 20 doublings in culture.

Certain embodiments are directed to methods of treatment wherein said cord tissue derived mesenchymal stem cell maintains a normal karyotype upon passaging

Certain embodiments are directed to methods of treatment wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Certain embodiments are directed to methods of treatment wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Certain embodiments are directed to methods of treatment wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

Certain embodiments are directed to methods of treatment wherein said umbilical cord tissue derived cells express markers selected from a group comprising of: a) TRA1-60; b) TRA1-81; c) SSEA3; d) SSEA4; and e) NANOG.

Certain embodiments are directed to methods of treatment wherein said umbilical cord tissue-derived cells are positive for alkaline phosphatase staining.

Certain embodiments are directed to methods of treatment wherein said umbilical cord tissue-derived cells are capable of differentiating into one or more lineages selected from a group comprising of; a) ectoderm; b) mesoderm, and; c) endoderm.

Certain embodiments are directed to methods of treatment wherein said bone marrow derived mesenchymal stem cells possess markers selected from a group comprising of: a) CD73; b) CD90; and c) CD105.

Certain embodiments are directed to methods of treatment wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.

Certain embodiments are directed to methods of treatment wherein said bone marrow derived mesenchymal stem cell is a mesenchymal stem cell progenitor cell.

Certain embodiments are directed to methods of treatment wherein said mesenchymal progenitor cells are a population of bone marrow mesenchymal stem cells enriched for cells containing STRO-1

Certain embodiments are directed to methods of treatment wherein said mesenchymal progenitor cells express both STRO-1 and VCAM-1.

Certain embodiments are directed to methods of treatment wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Certain embodiments are directed to methods of treatment wherein said bone marrow mesenchymal stem cells lack expression of CD14, CD34, and CD45.

Certain embodiments are directed to methods of treatment wherein said STRO-1 expressing cells are positive for a marker selected from a group comprising of: a) VACM-1; b) TKY-1; c) CD146 and; d) STRO-2

Certain embodiments are directed to methods of treatment wherein said bone marrow mesenchymal stem cell express markers selected from a group comprising of: a) CD13; b) CD34; c) CD56 and; d) CD117

Certain embodiments are directed to methods of treatment wherein said bone marrow mesenchymal stem cells do not express CD10.

Certain embodiments are directed to methods of treatment wherein said bone marrow mesenchymal stem cells do not express CD2, CD5, CD14, CD19, CD33, CD45, and DRII.

Certain embodiments are directed to methods of treatment wherein said bone marrow mesenchymal stem cells express CD13, CD34, CD56, CD90, CD117 and nestin, and which do not express CD2, CD3, CD10, CD14, CD16, CD31, CD33, CD45 and CD64.

Certain embodiments are directed to methods of treatment wherein said skeletal muscle stem cells express markers selected from a group comprising of: a) CD13; b) CD34; c) CD56 and; d) CD117

Certain embodiments are directed to methods of treatment wherein said skeletal muscle mesenchymal stem cells do not express CD10.

Certain embodiments are directed to methods of treatment wherein said skeletal muscle mesenchymal stem cells do not express CD2, CD5, CD14, CD19, CD33, CD45, and DRII.

Certain embodiments are directed to methods of treatment wherein said bone marrow mesenchymal stem cells express CD13, CD34, CD56, CD90, CD117 and nestin, and which do not express CD2, CD3, CD10, CD14, CD16, CD31, CD33, CD45 and CD64.

Certain embodiments are directed to methods of treatment wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Certain embodiments are directed to methods of treatment wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Certain embodiments are directed to methods of treatment wherein, said subepithelial umbilical cord derived mesenchymal stem cells express CD29, CD73, CD90, CD166, SSEA4, CD9, CD44, CD146, and CD105.

Certain embodiments are directed to methods of treatment wherein said subepithelial umbilical cord derived mesenchymal stem cells do not express CD45, CD34, CD14, CD79, CD106, CD86, CD80, CD19, CD117, Stro-1, and HLA-DR.

Certain embodiments are directed to methods of treatment wherein said subepithelial umbilical cord derived mesenchymal stem cells are positive for SOX2.

Certain embodiments are directed to methods of treatment wherein said subepithelial umbilical cord derived mesenchymal stem cells are positive for OCT4.

Certain embodiments are directed to methods of treatment wherein said subepithelial umbilical cord derived mesenchymal stem cells are positive for OCT4 and SOX2.

Certain embodiments are directed to methods of treatment wherein said stem cell derived products is stem cell conditioned media.

Certain embodiments are directed to methods of treatment wherein said stem cell derived products are stem cell derived microvesicles.

Certain embodiments are directed to methods of treatment wherein said stem cell derived products are stem cell derived exosomes.

Certain embodiments are directed to methods of treatment wherein said stem cell derived products are stem cell derived apoptotic vesicles.

Certain embodiments are directed to methods of treatment wherein said stem cell derived products are stem cell derived miRNAs.

Certain embodiments are directed to methods of treatment wherein said exosomes possess a size of between 30 nm and 150 nm.

Certain embodiments are directed to methods of treatment wherein said exosome possesses a size of between 2 nm and 200 nm, as determined by filtration against a 0.2 .mu.M filter and concentration against a membrane with a molecular weight cut-off of 10 kDa, or a hydrodynamic radius of below 100 nm as determined by laser diffraction or dynamic light scattering.

Certain embodiments are directed to methods of treatment wherein said exosome possesses a lipid selected from the group consisting of: a) phospholipids; b) phosphatidyl serine; c) phosphatidyl inositol; d) phosphatidyl choline; e) sphingomyelin; f) ceramides; g) glycolipid; h) cerebroside; i) steroids, and j) cholesterol.

Certain embodiments are directed to methods of treatment wherein said exosome possesses a lipid raft.

Certain embodiments are directed to methods of treatment wherein said exosome expresses antigenic markers on surface of said exosome, wherein said antigenic markers are selected from a group comprising of: a) CD9; b) CD63; c) CD81; d) ANXA2; e) ENO1; f) HSP90AA1; g) EEF1A1; h) YWHAE; i) SDCBP; j) PDCD6IP; k) ALB; l) YWHAZ; m) EEF2; n) ACTG1; o) LDHA; p) HSP90AB1; q) ALDOA; r) MSN; s) ANXA5; t) PGK1; and u) CFL1.

Certain embodiments are directed to methods of treatment wherein treatment of Leigh Syndrome is performed on one or more cell selected from a group comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, spiny neuronal cells, neuronal cells, dentate gyrus cells, cells of the brain medulla, cells of the brain stem, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Certain embodiments are directed to methods of treatment wherein at least one lithium compound or a pharmaceutically acceptable salt thereof, is administered.

Certain embodiments are directed to methods of treatment wherein said lithium compound, or a pharmaceutically acceptable salt thereof is selected from a group comprising of: a) lithium chloride; b) lithium bromide; c) lithium carbonate; d) lithium nitrate; e) lithium sulfate; f) lithium acetate; g) lithium lactate; h) lithium citrate; i) lithium aspartate; j) lithium gluconate; k) lithium malate; l) lithium ascorbate; m) lithium orotate; and n) lithium succinate.

Certain embodiments are directed to methods of treatment wherein at least one histone deacetylase inhibitor is added to culture of said stem cells at a concentration and frequency sufficient to enhance regenerative activity of said stem cell.

Certain embodiments are directed to methods of treatment wherein said histone deacetylase inhibitors are selected from a group comprising of: a) valproic acid; b) trichostatin A; c) suberoylanilide hydroxamic acid; d) oxamflatin; e) suberic bishydroxamic acid; f) m-carboxycinnamic acid bishydroxamic; g) pyroxamide; h) trapoxin A; i) apicidin; j) MS-27-275; k) butyric acid; and l) phenylbutyrate.

### DESCRIPTION OF THE INVENTION

In reviewing the detailed disclosure which follows, and the specification more generally, it should be borne in mind that all patents, patent applications, patent publications, technical publications, scientific publications, and other references referenced herein are hereby incorporated by reference in this application, in their entirety to the extent not inconsistent with the teachings herein. It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise

For the practice of the invention, a preferred embodiment is the administration of mesenchymal stem cells (MSC) intravenously at concentrations sufficient to treat Leigh Syndrome. Without being bound to theory, administration of said mesenchymal stem cells may be in the form of cells themselves, extracts of the cells, lysates, or nucleic acid compositions, said administration, while possessing ability to reduce and/or reverse pathology of Leigh Syndrome, may function through means including restoration of mitochondrial enzymes, protection of neural cells from cellular death, stimulation of neural regeneration, and/or providing transfer of genetic material.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In a particularly preferred embodiment mes are cultured in the cell culture system which is a cell culture system, comprising a cell culture medium, preferably in a culture vessel, in particular a cell culture medium supplemented with a substance suitable and determined for culturing the cells in a manner so as to endow ability to prevent, inhibit progression, or reverse Leigh Syndrome.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage [23, 24]. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow [25-29], adipose tissue [30, 31], amniotic fluid [32, 33], endometrium [34-37], trophoblast-associated tissues [38], human villous trophoblasts [39], cord blood [40], Wharton jelly [41-43], umbilical cord tissue [44], placenta [45], amniotic tissue [46-48], derived from pluripotent stem cells [49-53], and tooth.

In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion.

Furthermore, as used herein, in some contexts, “MSC” includes cells described in the literature as bone marrow stromal stem cells (BMSSC) [54], marrow-isolated adult multipotent inducible cells (MIAMI) cells [55, 56], multipotent adult progenitor cells (MAPC) [57-60], MultiStem®, Prochymal [61-65], remestemcel-L [66], Mesenchymal Precursor Cells (MPCs) [67], Dental Pulp Stem Cells (DPSCs) [68], PLX cells [69], Ixmyelocel-T [70], NurOwn™ [71], Stemedyne™-MSC, Stempeucel® [72, 73], HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs) [74].

In accordance with the presently disclosed invention, the word “comprising” is synonymous with “including,” “having,” “containing,” or “characterized by.” These terms are inclusive and open-ended and do not exclude additional, unrecited elements or method steps.

In accordance with the presently disclosed invention, the phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

In accordance with the presently disclosed invention, the phrase “consisting essentially of” limits the scope of a claim to the specified materials or steps, plus those that do not materially affect the basic and novel characteristic(s) of the claimed subject matter. It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

In accordance with the presently disclosed invention, the word “dedifferentiation” describes the process of a cell “going back” in developmental time. In this respect, a dedifferentiated cell acquires one or more characteristics previously possessed by that cell at an earlier developmental time point. An example of dedifferentiation is the temporal loss of epithelial cell characteristics during wounding and healing. Dedifferentiation can occur, in degrees. In the afore-mentioned example of wound healing, dedifferentiation progresses only slightly before the cells redifferentiate to recognizable epithelia. A cell that has greatly dedifferentiated, for example, is one that resembles a stem cell. Dedifferentiated cells can either remain dedifferentiated and proliferate as a dedifferentiated cell; redifferentiate along the same developmental pathway from which the cell had previously dedifferentiated; or redifferentiate along a developmental pathway distinct from which the cell had previously dedifferentiated. Within the context of the present invention, a dedifferentiated mesenchymal stem cell possesses enhanced plasticity and ability to differentiate, or “redifferentiate” into other cells. The dedifferentiated state of the treated cell, which in the current invention is a mesenchymal stem cell, can be verified by increased expression of one or more genes selected from the group consisting of alkaline phosphatase (ALP), OCT4, SOX2, human telomerase reverse transcriptase (hERT) and SSEA-4. That is, the somatic cells introduced with the reprogramming gene are treated with the functional peptide, and then an initial process in which a colony is generated in the dedifferentiation process is observed through alkaline phosphatase staining (AP staining), and furthermore, expression of Oct4 is verified by immunofluorescence (IF) using an Oct4 antibody.

In accordance with the invention presented herein, the term “reprogramming” preferably means remodelling, in particular erasing and/or remodelling, epigenetic marks of a cell such as DNA methylation, histon methylation or activating genes by inducing transcription factor signal systems as for oct4. In particular, the reprogramming of the present invention provides at least one dedifferentiated and/or rejuvenated cell, in particular provides a cell having the characteristic of a multipotent, in particular pluripotent stem cell. Thus, in case the cell to be reprogrammed is cells which already have a multipotent or pluripotent character, the present invention is able to maintain these cells by the reprogramming of the present invention in their multi- or pluripotent state for a prolonged period of time. In case the cells to be reprogrammed are in an aged or differentiated state, the present invention allows the dedifferentiation into a multipotent or pluripotent stem cell. In a particularly preferred embodiment, multipotent cells may be reprogrammed to become pluripotent cells. The cells of the invention are particularly mesenchymal stem cells which are to be reprogrammed.

In accordance with the invention presented herein, the word “stem cell”, refers to any self-renewing pluripotent cell or multipotent cell or progenitor cell or precursor cell that is capable of differentiating into one or multiple cell types. Stem cells are thus cells able to differentiate into one or more than one cell type and have preferably an unlimited growth potential. Stem cells include those that are capable of differentiating into cells of osteoblast lineage, a mesenchymal cell lineage (e. g. bone, cartilage, adipose, muscle, stroma, including hematopoietic supportive stroma, and tendon). “Differentiate” or “differentiation”, as used herein, refers to the process by which precursor or progenitor cells (i. e., stem cells) differentiate into specific cell types, e. g., osteoblasts. Differentiated cells can be identified by their patterns of gene expression and cell surface protein expression. “Dedifferentiate” or “dedifferentiation”, as used herein, refers to the process by which lineage-committed cells (e. g., myoblasts or osteoblasts) reverse their lineage commitment and become precursor or progenitor cells (i. e., multipotent or pluripotent stem cells). Dedifferentiated cells can for instance be identified by loss of patterns of gene expression and cell surface protein expression associated with the lineage committed cells.

In accordance with the invention presented herein, the words “cell culture” and “culturing of cells” refer to the maintenance and propagation of cells and preferably human, human-derived and animal cells in vitro.

In accordance with the invention presented herein, the words “Cell culture medium” is used for the maintenance of cells in culture in vitro. For some cell types, the medium may also be sufficient to support the proliferation of the cells in culture. A medium according to the present invention provides nutrients such as energy sources, amino acids and anorganic ions. Additionally, it may contain a dye like phenol red, sodium pyruvate, several vitamins, free fatty acids, antibiotics, anti-oxidants and trace elements. For culturing the mesenchymal stem cells that are dedifferentiated into stem cells, or stem cell-like cells according to the present invention any standard medium such as Iscove's Modified Dulbecco's Media (IMDM), alpha-MEM, Dulbecco's Modified Eagle Media (DMEM), RPMI Media and McCoy's Medium is suitable before reprogramming. Ones the cells have been reprogrammed, they can in a preferred embodiment be cultured in embryonic stem cell medium.

In accordance with the invention presented herein, the word “Transfection” refers to a method of gene delivery that introduces a foreign nucleotide sequences (e.g. DNA/RNA or protein molecules) into a cell preferably by a viral or non-viral method. In preferred embodiments according to the present invention foreign DNA/RNA/proteins are introduced to a cell by transient transfection of an expression vector encoding a polypeptide of interest, whereby the foreign DNA/RNA/proteins is introduced but eliminated over time by the cell and during mitosis. By “transient transfection” is meant a method where the introduced expression vectors and the polypeptide encoded by the vector, are not permanently integrated into the genome of the host cell, or anywhere in the cell, and therefore may be eliminated from the host cell or its progeny over time. Proteins, polypeptides, or other compounds can also be delivered into a cell using transfection methods.

In accordance with the invention presented herein, the concept of identifying the “sufficient period of time” to allow stable expression of the at least one gene regulator in absence of the reprogramming agent and the “sufficient period of time” in which the cell is to be maintained in culture conditions supporting the transformation of the desired cell is within the skill of those in the art. The sufficient or proper time period will vary according to various factors, including but not limited to, the particular type and epigenetic status of cells (e.g. the cell of the first type and the desired cell), the amount of starting material (e.g. the number of cells to be transformed), the amount and type of reprogramming agent(s), the gene regulator(s), the culture conditions, presence of compounds that speed up reprogramming (ex, compounds that increase cell cycle turnover, modify the epigenetic status, and/or enhance cell viability), etc. In various embodiments the sufficient period of time to allow a stable expression of the at least one gene regulator in absence of the reprogramming agent is about 1 day, about 2-4 days, about 4-7 days, about 1-2 weeks, about 2-3 weeks or about 3-4 weeks. In various embodiments the sufficient period of time in which the cells are to be maintained in culture conditions supporting the transformation of the desired cell and allow a stable expression of a plurality of secondary genes is about 1 day, about 2-4 days, about 4-7 days, or about 1-2 weeks, about 2-3 weeks, about 3-4 weeks, about 4-6 weeks or about 6-8 weeks. In preferred embodiments, at the end of the transformation period, the number of transformed desired cells is substantially equivalent or even higher than an amount of cells a first type provided at the beginning.

Said MSC may be expanded and utilized by administration themselves, or may be cultured in a growth media in order to obtain conditioned media, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Mesenchymal stem cells (“MSC”) may be derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+SH4.sup.+CD29.sup.+CD44.sup.+CD71.sup.+CD90.sup.+CD106.sup.+CD120a.sup.+CD124.sup.+CD14.sup.-CD34.sup.-CD45.sup.−)). The invention teaches the use of various mesenchymal stem cells

In one embodiment MSC donor lots are generated from umbilical cord tissue. Means of generating umbilical cord tissue MSC have been previously published and are incorporated by reference [40, 43, 75-79]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C—X—C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C—X—C motif) ligand 1 (melonoma growth stimulating activity, alpha); chemokine (C—X—C motif) ligand 6 (granulocyte chemotactic protein 2); chemokine (C—X—C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In one embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIB ERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest. While the use of enzyme is presently preferred, it is not required for isolation methods as provided herein. Methods based on mechanical separation alone may be successful in isolating the instant cells from the umbilicus as discussed above. The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells. Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 200 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10 [symbol] 10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2 [symbol] 10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1 [symbol] 10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1 [symbol] 10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In one embodiment, hematopoietic stem cells are CD34+ cells isolated from the peripheral blood, bone marrow, or umbilical cord blood. Specifically, the hematopoietic stem cells may be derived from the blood system of mammalian animals, include but not limited to human, mouse, rat, and these hematopoietic stem cells may be harvested by isolating from the blood or tissue organs in mammalian animals. Hematopoietic stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and cell proliferation and differentiation,” issued Jan. 1, 2002, which are hereby incorporated by reference in their entireties. In some embodiments, stem cells obtained from the donor are cultured in order to expand the population of stem cells. In other preferred embodiments, stem cells collected from donor sources are not expanded using such techniques. Standard methods can be used to cyropreserve the stem cells.

In some embodiments of the invention, where there are risks associated with particular types of stem cells, for example, pluripotent stem cells, said stem cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of stem cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of stem cells are known to those of skill in the art and are described, for example, in Chang, P., et al., 1999; Matthew, H. W., et al., 1991; Yanagi, K., et al., 1989; Cal Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of stem cells. Certain embodiments incorporate stem cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, stem cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

In some embodiments of the invention, mesenchymal stem cells are cultured with substances capable of maintaining said mesenchymal stem cells in an immature state, and/or maintaining high expression of genes/mitochondria necessary to prevent, inhibit, and/or reverse Leigh Syndrome. Said substances are selected from the group consisting of reversin, cord blood serum, lithium, a GSK-3 inhibitor, resveratrol, pterostilbene, selenium, a selenium-containing compound, EGCG ((−)-epigallocatechin-3-gallate), valproic acid and salts of valproic acid, in particular sodium valproate. In one embodiment of the present invention, a concentration of reversin from 0.5 to 10 .mu.M, preferably of 1 .mu.M is added to the mesenchymal stem cell culture. In a furthermore preferred embodiment the present invention foresees to use resveratrol in a concentration of 10 to 100 .mu.M, preferably 50 .mu.M. In a furthermore preferred embodiment the present invention foresees to use selenium or a selenium containing compound in a concentration from 0.05 to 0.5 .mu.M, preferably of 0.1 .mu.M. In another embodiment, cord blood serum is added at a concentration of 0.1%-20% volume to the volume of tissue culture media. In furthermore preferred embodiment the present invention foresees to use EGCG in a concentration from 0.001 to 0.1 .mu.M, preferably of 0.01 .mu.M. In a furthermore preferred embodiment the present invention foresees to use valproic acid or sodium valproate in a concentration from 1 to 10 .mu.M, in particular of 5 .mu.M. In some embodiments, mesenchymal stem cells are retrodifferentiated to possess higher expression of regenerative genes. Said retrodifferentiation may be achieved by cytoplasmic transfer, transfection of cytoplasm, or cell fusion with a stem cell possessing a higher level of immaturity, said stem cells including pluripotent stem cells. In such culture/coculture procedures, the cell culture medium comprises, optionally in combination with one or more of the substances specified above, at least one transient proteolysis inhibitor. The use of at least one proteolysis inhibitor in the cell culture medium of the present invention increases the time the reprogramming proteins derived from the mRNA or any endogenous genes will be present in the cells and thus facilitates in an even more improved way the reprogramming by the transfected mRNA derived factors. The present invention uses in a particular embodiment a transient proteolysis inhibitor a protease inhibitor, a proteasome inhibitor and/or a lysosome inhibitor. In an embodiment the proteosome inhibitor is selected from the group consisting of MG132, TMC-95A, TS-341 and MG262. In a furthermore preferred embodiment the protease inhibitor is selected from the group consisting of aprotinin, G-64 and leupeptine-hemisulfat. In a furthermore preferred embodiment the lysosomal inhibitor is ammonium chloride. In one embodiment the present invention also foresees a cell culture medium comprising at least one transient inhibitor of mRNA degradation. The use of a transient inhibitor of mRNA degradation increases the half-life of the reprogramming factors as well. Another embodiment of the present invention a condition suitable to allow translation of the transfected reprogramming mRNA molecules in the cells is an oxygen content in the cell culture medium from 0.5 to 21%. More particular, and without wishing to be bound to the theory, oxygen is used to further induce or increase Oct4 by triggering Oct4 via Hif1a, in these situations concentrations of oxygen lower than atmospheric concentration are used, and can be ranging from 0.1% to 10%. In a preferred embodiment conditions that are suitable to support reprogramming of the cells by the mRNA molecules in the cells are selected; more particularly, these conditions require a temperature from 30 to 38.degree. C., preferably from 31 to 37.degree. C., most preferably from 32 to 36.degree. C. The glucose content of the medium is in a preferred embodiment of the present invention below 4.6 g/1, preferably below 4.5 g/1, more preferably below 4 g/1, even more preferably below 3 g/1, particularly preferably below 2 g/I and most preferably it is 1 g/1. DMEM media containing 1 g/l glucose being preferred for the present invention are commercially available as “DMEM low glucose” from companies such as PAA, Omega Scientific, Perbio and Biosera. More particular, and without wishing to be bound to the theory, high glucose conditions adversely support aging of cells (methylation, epigenetics) in vitro which may render the reprogramming difficult. In a furthermore preferred embodiment of the present invention the cell culture medium contains glucose in a concentration from 0.1 g/l to 4.6 g/1, preferably from 0.5 g/l to 4.5 g/l and most preferably from 1 g/l to 4 g/1.

In the generation of mesenchymal stem cells useful for treatment of Leigh Syndrome, it may be advantageous to endow cells with a particular phenotype to possess sufficient “potency” to stimulate regenerative processes. Donor cells that are useful for the invention are dependent on the desired use of the generated cell, along with the specific pathology of the Leigh Syndrome patient. For example, in one embodiment, RNA or mRNA is extracted to achieve pluripotency in the ‘target’ cells include by way of example oocytes, inducible pluripotent stem cells, and somatic cell nuclear transfer generated pluripotent cells, from any species including human and vertebrates such as amphibians, fish, and mammals. In some examples, donor cells are transfected to overexpress genes that are deficient in Leigh Syndrome. Such genes include: a) AIFM1; b) BCS1L; c) BTD; d) C12orf65; e) COX10; f) COX15; g) DLAT; h) DLD; i) EARS2; j) ECHS1; k) ETHE1; l) FARS2; m) FBXL4; n) FOXRED1; o) GFM1; p) GFM2; q) GTPBP3; r) HIBCH; s) IARS2; t) LIAS; u) LIPT1; v) LRPPRC; w) MT-ATP6; x) MT-CO3; y) MT-ND1; z) MT-ND2; aa) MT-ND3; ab) MT-ND4; ac) MT-ND4; ad) MT-ND5; ae) MG-ND6; af) MT-TI; ag) MT-TK; ah) MT-TL1; ai) MT-TV; aj) MT-TW; ak) MTFMT; al) NARS2; am) NDUFA1; an) NDUFA2; ao) NDUFA4; ap) NDUFA9; aq) NDUFA10; ar) NDUFA11; as) NDUFA12; at) NDUFAF2; au) NDUFAF5; aw) NDUFAF6; ax) NDUFS1; ay) NDUFS2; az) NDUFS3; ba) NDUFS4; bb) NDUFS7; bc) NDUFS8; bd) NDUFV1; be) NDUFV2; bf) PDHA1; bg) PDHB; bh) PDHX; bi) PDSS2; bj) PET100; bk) PNPT1; bl) POLG; bm) SCO2; bn) SDHA; bo) SDHAF1; bp) SERAC1; bq) SLC19A3; br) SLC25A19; bs) SUCLA2; bt) SUCLG1; bu) SURF1; by) TACO1; bw) TPK1; bx) TRMU; by) TSFM; bz) TTC19; and ca) UQCRQ.

In some embodiments, autologous mesenchymal stem cells from Leigh Syndrome patients are used as target cells, which are subsequently transfected with therapeutic genes and retrodifferentiated to achieve higher degree of transfection efficacy and gene repair. Examples of recipient or target cells into which RNA or mRNA can be introduced to achieve pluripotency or transdifferentiation in the ‘target’ cells are mesenchymal stem cells. Various sources of mesenchymal stem cells may be used, depending on tissue and age. Examples of somatic cells which may be used as the donor cell for transdifferentiation include any cell type that is desired for cell therapies are cells relevant to pathology of Leigh Syndrome. The current invention further provides dedifferentiation of target cells using total RNA or mRNA. The mRNA or total RNA used to effect dedifferentiation is preferably isolated from cells that are either pluripotent or which are capable of turning into pluripotent cells (oocyte). Examples thereof include by way of example Ntera cells, human or other ES cells, primordial germ cells, and blastocysts. Alternatively the RNA used to effect dedifferentiation may comprise mRNA encoding specific transcription factors. The total RNA or mRNA's may be delivered into target cells by different methods including e.g., electroporation, liposomes, and mRNA injection. Target cells into which RNA's are introduced and which are to be dedifferentiated according to the invention are cultured in a medium containing one or more constituents that facilitates transformation of cell phenotype. These constituents include by way of example epigenetic modifiers such as DNA demethylating agents, HDAC inhibitors, histone modifiers; and cell cycle manipulation and pluripotent or tissue specific promoting agents such as helper cells which promote growth of pluripotent cells, growth factors, hormones, and bioactive molecules. Examples of DNA methylating agents include 5-azacytidine (5-aza), MNNG, 5-aza, N-methyl-N′-nitro-N-nitrosoguanidine, temozolomide, procarbazine, et al. Examples of methylation inhibiting drugs agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA. Examples of histone deacetylase (“HDAC”) inhibitor is selected from a group consisting of hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Preferred examples of histone modifiers include PARP, the human enhancer of zeste, valproic acid, and trichostatine. Particular constituents that the inventors utilize in a preferred media in order to facilitate RNA transformation and dedifferentiation of the RNA comprising target cells into pluripotent cells include trichostatine, valproic acid, zebularine and 5-aza. Target cells into which RNA is introduced are cultured for a sufficient time in media that promotes RNA transformation until dedifferentiated cells (pluripotent) cells are obtained. In some instances this methodology may be combined with other methods and treatments involved in the epigenetic status of the recipient or target cell such as the exposure to DNA and histone demethylating agents, histone deacetylase inhibitors, and/or histone modifiers. This invention therefore describes a method of changing the fate or phenotype of cells. By using epigenetic modifications, the subject methods can dedifferentiate or transdifferentiate cells. This invention is aimed to solve the problem of immuno-rejection which is evident when incompatible cells/tissues are used for transplantation. Cells from one patient can be transformed into a different type of cell allowing for the derivation of cells needed for the treatment of a particular disease the patient is suffering from. One of the types of cells that can be produced by this invention is pluripotent stem cells. This invention also offers an opportunity to the research community to study the mechanisms involved in cell differentiation and disease progression.

In addition, the recipient cells may be cultured under different conditions that enhance reprogramming efficiency such as co-culture of the RNA transfected cells with other cell types, conditioned medias, and by the supplementation of the culture medium with other biological agents such as growth factors, hormones, vitamins, etc. which enhance growth and maintenance of the cultured cells. In one embodiment of the invention, mesenchymal stem cells are treated with one or more “Inhibitor(s) of DNA methylation”. This term refers to an agent that can inhibit DNA methylation. DNA methylation inhibitors have demonstrated the ability to restore suppressed gene expression. Suitable agents for inhibiting DNA methylation include, but are not limited to 5-azacytidine, 5-aza-2-deoxycytidine, 1-.beta.-D-arabinofuranosil-5-azacytosine, and dihydro-5-azacytidine, and zebularine (ZEB), BIX (histone lysine methyltransferase inhibitor), and RG108. Concentration of DNA methylation inhibitors, as well as duration of exposure, is dependent on ability to induce expansion of plasticity.

For practice of the invention, inhibitors of acetylation are used in culture of mesenchymal stem cells. This term refers to an agent that prevents the removal of the acetyl groups from the lysine residues of histones that would otherwise lead to the formation of a condensed and transcriptionally silenced chromatin. Histone deacetylase inhibitors fall into several groups, including: (1) hydroxamic acids such as trichostatin (A) [4-7], (2) cyclic tetrapeptides, (3) benzamides, (4) electrophilic ketones, and (5) aliphatic acid group of compounds such as phenylbutyrate and valporic acid. Suitable agents to inhibit histone deacetylation include, but are not limited to, valporic acid (VPA) [8-19], phenylbutyrate and Trichostatin A (TSA). One example, in the area of mesenchymal stem cells, of valproic acid enhancing pluripotency and therapeutic properties is provided by Killer et al. who showed that culture of cells with valproic acid enhanced immune regulatory and metabolic properties of mesenchymal stem cells. The culture systems described, as well as means of assessment, are provided to allow one of skill in the art to have a starting point for the practice of the current invention [20, 21]. Without being bound to theory, valproic acid in the context of the current invention may be useful to increasing in vitro proliferation of dedifferentiated mesenchymal stem cells while preventing senescence associated stress. For example, Zhai et al showed that in an in vitro pre-mature senescence model, valproic acid treatment increased cell proliferation and inhibited apoptosis through the suppression of the p16/p21 pathway. In addition, valproic acid also inhibited the G2/M phase blockage derived from the senescence stress [22].

In some embodiments of the invention, small RNAs that act as small activating RNA (saRNA) which induce activation of OCT4 expression are applied to mesenchymal stem cell to induce dedifferentiation. In some cases this is combined with histone deacetylase inhibitors and/or GSK3 inhibitors and/or DNA methyltransferase inhibitors, in order to induce a dedifferentiated phenotype in the mesenchymal stem cells. Such mesenchymal stem cells that have been dedifferentiated can subsequently be used as a source of cells for differentiation into therapeutic cells. Small RNAs that act as small activating RNAs of the OCT4 promotor are described in the following publications [23-28].

In some embodiments, mesenchymal stem cells are transfected with miRNA and dedifferentiated before differentiating into cells of relevance to Leigh Syndrome. Mesenchymal stem cells may be purchased from companies such as Lonza, and cultured in DMEM medium (Invitrogen, Life Technologies Ltd) containing 10% fetal bovine serum (PAA), 2 mM L-glutamine (Invitrogen, Life Technologies Ltd), lx MEM non-essential amino acid solution, lx Penicillin/Streptomycin (PAA) and β-mercaptoethanol (Sigma-Aldrich). Mesenchymal stem cells may be transduced using lentiviral particles containing hsa-miR-145-5p inhibitor (Genecopoeia) at MOI=40 in the presence of 5 μg/ml Polybrene (Sigma-Aldrich). Transduced cells were selected for Hygromycin resistance (50-75 μg/ml). For transient miR-145 inhibition, 1×105 mesenchymal stem cells are transfected with 100 pmoles miR-145 mirVana® miRNA inhibitor (Life Technologies Ltd) using Neon transfection system (Invitrogen). Transfection is carried out by two 1600 V pulses for 20 ms. For reprogramming, cells are transduced using CytoTune®-iPS Sendai Reprogramming Kit (Product number A1378001) (Life Technologies Ltd) according to manufacturer's instructions. The efficiency of mesenchymal stem cell dedifferentiation can be assessed by alkaline phosphatase (AP) activity staining using Alkaline Phosphatase Blue Substrate (Sigma-Aldrich) and by TRA-1-60 expression, as determined indirect immunofluorescence. Cells are washed with PBS, fixed by 4% paraformaldehyde for 10 minutes at room temperature, washed again with PBS, and incubated overnight at 4° C. with primary antibody against TRA-1-60 (MAB4360, Merck Millipore). Then cells are washed three times with PBS and incubated with Alexa 488-conjugated secondary antibody and observed under fluorescent microscope [29].

One of skill in the art will understand that there exist numerous alternative steps for facilitating cell reprogramming which may be applied to mesenchymal stem cells. These methods include the destabilizing the cell's cytoskeletal structure (for example, by exposing the cell to cytochalasin B), loosening the chromatin structure of the cell (for example, by using agents such as 5 azacytidine (5-Aza) and Valproic acid (VPA) or DNA demethylator agents such as MBD2), transfecting the cell with one or more expression vector(s) containing at least one cDNA encoding a dedifferentiating factor(for example, OCT4, SOX-2, NANOG, or KLF), using an appropriate medium for the desired cell of a different type and an appropriate differentiation medium to induce dedifferentiation of the mesenchymal stem cells, inhibiting repressive pathways that negatively affects induction into commitment the desired cell of a different type, growing the cells on an appropriate substrate for the desired cell of a different type, and growing the cells in an environment that the desired cell of a different type (or “-like” cell) would be normally exposed to in vivo such as the proper temperature, pH and low oxygen environment (for example about 2-5% O.sub.2). In various embodiments, the invention encompasses these and other related methods and techniques for facilitating cell reprogramming/dedifferentiation.

Treatment of Leigh Syndrome may require various combinatorial approaches within the practice of the current invention. Specifically, administration of stem cell derived factors, including lysates, conditioned media, microvesicles, apoptotic bodies, mitochondria or exosomes. In one embodiment of the invention, exosomes are purified from mesenchymal stem cells by obtaining a mesenchymal stem cell conditioned medium, concentrating the mesenchymal stem cell conditioned medium, subjecting the concentrated mesenchymal stem cell conditioned medium to size exclusion chromatography, selecting UV absorbent fractions at 220 nm, and concentrating fractions containing exosomes.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of .about.1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in mesenchymal stem cells or mesenchymal stem cell conditioned medium (MSC-CM), such as a protein characteristic or specific to the MSC or MSC-CM. They may comprise RNA, for example miRNA. Said particles may possess one or more genes or gene products found in MSCs or medium which is conditioned by culture of MSCs. The particle may comprise molecules secreted by the MSC. Such a particle, and combinations of any of the molecules comprised therein, including in particular proteins or polypeptides, may be used to supplement the activity of, or in place of, the MSCs or medium conditioned by the MSCs for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes that the MSC or MSC-CM may be put to use.

In one embodiment, MSC exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

In some embodiments of the invention, treatment of Leigh Syndrome is performed by administration of cellular lysate from regenerative cells. Said regenerative cells may be mesenchymal stem cells, in one preferred embodiment said mesenchymal stem cells are derived from the umbilical cord. Derivation of mesenchymal stem cells from umbilical cord/Wharton's Jelly for clinical applications are described in the art and incorporated by reference [80-88]. For practice of the invention, xenogeneic free media may be used to grow mesenchymal stem cells to reduce possibility of sensitization from components such as fetal calf serum [44, 89-95]. In some embodiments of the invention, mesenchymal stem cells are pretreated using ways of enhancing regenerative activity, said means include treatment with histone deacetylase inhibitors such as valproic acid, GSK-3 inhibitors such as lithium [96-101], culture under hypoxia, and treatment with carbon monoxide [102].

In some embodiments, mesenchymal stem cells may be synchronized in G2 by incubating the cells in the presence of aphidicolin to arrest them in S phase and then washing the cells three times by repeated centrifugation and resuspension in phosphate buffered saline (PBS), as described herein. The cells are then incubated for a length of time sufficient for cells to enter G2 phase. For example, cells with a doubling time of approximately 24 hours, may be incubated for between 6 and 12 hours to allow them to enter G2 phase. For cells with shorter or longer doubling times, the incubation time may be adjusted accordingly. In some embodiments of the invention, mesenchymal stem cells may be synchronized in mitosis by incubating them in 0.5 .mu.g/ml nocodazole for 17-20 hours, and the mitotic cells are detached by vigorous shaking. The detached G1 phase doublets may be discarded, or they may be allowed to remain with the mitotic cells which constitute the majority (over 80%) of the detached cells. The harvested detached cells are centrifuged at 500 g for 10 minutes in a 10 ml conical tube at 4.degree. C. Synchronized or unsynchronized cells may be harvested using standard methods and washed by centrifugation at 500 g for 10 minutes in a 10 ml conical tube at 4.degree. C. The supernatant is discarded, and the cell pellet is resuspended in a total volume of 50 ml of cold PBS. The cells are centrifuged at 500 g for 10 minutes at 4.degree. C. This washing step is repeated, and the cell pellet is resuspended in approximately 20 volumes of ice-cold interphase cell lysis buffer (20 mM Hepes, pH 8.2, 5 mM MgCl.sub.2, 1 mM DTT, 10 pM aprotinin, 10 pM leupeptin, 10 pM pepstatin A, 10 pM soybean trypsin inhibitor, 100 pM PMSF, and optionally 20 pg/ml cytochalasin B). The cells are sedimented by centrifugation at 800 g for 10 minutes at 4.degree. C. The supernatant is discarded, and the cell pellet is carefully resuspended in no more than one volume of interphase cell lysis buffer. The cells are incubated on ice for one hour to allow swelling of the cells. The cells are then lysed by either sonication using a tip sonicator or Dounce homogenization using a glass mortar and pestle. Cell lysis is performed until at least 90% of the cells and nuclei are lysed, which may be assessed using phase contrast microscopy. Duration and power of sonication required to lyse at least 90% of the cells and nuclei may vary depending on the type of cell used to prepare the extract.

In some embodiments, the cell lysate is placed in a 1.5-ml centrifuge tube and centrifuged at 10,000 to 15,000 g for 15 minutes at 4.degree. C. using a table top centrifuge. The tubes are removed from the centrifuge and immediately placed on ice. The supernatant is carefully collected using a 200 .mu.l pipette tip, and the supernatant from several tubes is pooled and placed on ice. This supernatant is the cytoplasmic extract. This cell extract may be aliquoted into 20 pl volumes of extract per tube on ice and immediately flash-frozen on liquid nitrogen and stored at 80.degree. C. until use. Alternatively, the cell extract is placed in an ultracentrifuge tube on ice (e. g., fitted for an SW55 Ti rotor; Beckman). If necessary, the tube is overlayed with mineral oil to the top. The extract is centrifuged at 200,000 g for three hours at 4.degree. C. to sediment membrane vesicles contained in the cytoplasmic extract. At the end of centrifugation, the oil is discarded. The supernatant is carefully collected, pooled if necessary, and placed in a cold 1.5 ml tube on ice.

In other embodiments, mesenchymal stem cell lysate is generated by rinsing cells 3-4 times with PBS, and culture medium, such as alpha-MEM or DMEM/F12 (Gibco) is added without additives or serum. 12-24 hours later, the cells are washed twice with PBS and harvested, preferably scraped with a rubber policeman and collected in a 50 ml Falcon tube (Becton Dickinson). Then cells are washed and resuspended in ice-cold cell lysis buffer (20 mM HEPES, pH 8.2, 50 mM NaCl, 5 mM MgCl.sub.2, 1 mM dithiothreitol and a protease inhibitor cocktail), sedimented at 400 g and resuspended in one volume of cell lysis buffer. Cells are sonicated on ice in 200 .mu.l aliquots using a sonicator fitted with a 2-mm diameter probe until all cells and nuclei are lysed, as can be judged by phase contrast microscopy. The lysate is centrifuged at 10,000-14,000 g, 15-30 minutes at 4.degree. C. to pellet the coarse material and any potentially remaining non-lysed cell. The supernatant is aliquoted, frozen and stored in liquid nitrogen or immediately used. Protein concentration of the extract is analyzed by Bradford assay, pH is adjusted to around 7.0.+−0.0.4 and oslolarity is adjusted to −300 mOsm prior to use, in necessary, (by diluting with water).

In addition to cell lysate, conditioned media from cells may be utilized. Both cell lysate and conditioned media may be administered intranasally through an aerosolation means, or may be administered orally, intravenously, subcutaneously, intrarectally, intramuscularly, or sublingually.

Conditioned media may be generated in order to concentrated secreted factors, or may be utilized as a source of exosomes. In some embodiments, exosomes are concentrated by means of ultracentrifugation, chromatography, or based on adhesion to substrates.

### EXAMPLES

### Example 1

A 5 year old male, diagnosed with Leigh Syndrome based on clinical observations and genetic mitochondrial mutations, was recruited for treatment based on physician recommendation. Prior to treatment, the patient had no ability to walk and limited speech. A high degree of nystagmus with 90 degree eye discoordination was present. Patient had to be hospitalized approximately once every two months prior to treatment.

The patient was administered lysate derived from umbilical mesenchymal stem cells intranasally for 2 treatments, separated by one day. The patient also received intravenous administration of umbilical cord derived mesenchymal stem cells for 4 consecutive days. Subsequent to receiving treatment the patient was able to walk, nystagmus markedly improved. Reduced need for hospitalization was observed. Improvements were present at 6 months after treatment. Additionally, improvements in energy levels after treatment were observed.

### Example 2

A 6 year old male diagnosed with Leigh Syndrome possessing the Surf1 mutation was recruited for treatment based on physician recommendation. The patient had a history of frequent vomiting and low energy.

The patient was administered lysate derived from umbilical mesenchymal stem cells intranasally for 2 treatments, separated by one day. The patient also received intravenous administration of umbilical cord derived mesenchymal stem cells for 4 consecutive days. Subsequent to receiving treatment the patient had improved ability to walk, reduced vomiting episodes and reduced need for hospitalization was observed. The patient was free from vomiting for 4 months subsequent to treatment.

### Example 3

A four year old male diagnosed with Leigh Syndrome caused by a homozygous mutation in the gene C120RF65 was treated. Prior to treatment the patient had a general decline in health, being hospitalized multiple times, had quit walking, and was eating through a feeding tube. Patient was treated with 40 million stem cells. Jaxson soon began walking again with assistance, and in the second month without assistance. Improvements were made in fine motor skills, speech, and appetite. These improvements lasted four months after treatment, but by the fifth month, the patient had lost the gains he had made and began using a walker for assisted walking. About 7 months after his first round of treatment, the patient underwent a 2^{nd} round of stem cell treatments, resulting in similar positive results. A 3^{rd} round of stems cells was administered about 3 months after the 2^{nd} round, with no decline in improvement in between. Similar positive results were seen after the 3^{rd} round of treatment.

### REFERENCES

1. Thorburn, D. R., J. Rahman, and S. Rahman, Mitochondrial DNA-Associated Leigh Syndrome and NARP, in GeneReviews(R), M. P. Adam, et al., Editors. 1993: Seattle (Wash.).
2. Bonfante, E., et al., The neuroimaging of Leigh syndrome: case series and review of the literature. Pediatr Radiol, 2016. 46(4): p. 443-51.
3. Ogawa, E., et al., Clinical validity of biochemical and molecular analysis in diagnosing Leigh syndrome: a study of 106 Japanese patients. J Inherit Metab Dis, 2017.
4. de Haas, R., F. G. Russel, and J. A. Smeitink, Gait analysis in a mouse model resembling Leigh disease. Behav Brain Res, 2016. 296: p. 191-8.
5. Wang, M., et al., Mitochondrial complex I deficiency leads to the retardation of early embryonic development in Ndufs4 knockout mice. PeerJ, 2017. 5: p. e3339.
6. Kruse, S. E., et al., Mice with mitochondrial complex I deficiency develop a fatal encephalomyopathy. Cell Metab, 2008. 7(4): p. 312-20.
7. Quintana, A., et al., Complex I deficiency due to loss of Ndufs4 in the brain results in progressive encephalopathy resembling Leigh syndrome. Proc Natl Acad Sci USA, 2010. 107(24): p. 10996-1001.
8. De Vivo, D. C., The expanding clinical spectrum of mitochondrial diseases. Brain Dev, 1993. 15(1): p. 1-22.
9. Ortigoza-Escobar, J. D., et al., Ndufs4 related Leigh syndrome: A case report and review of the literature. Mitochondrion, 2016. 28: p. 73-8.
10. Johnson, S. C., et al., mTOR inhibition alleviates mitochondrial disease in a mouse model of Leigh syndrome. Science, 2013. 342(6165): p. 1524-8.
11. Ito, T. K., et al., Hepatic S6K1 Partially Regulates Lifespan of Mice with Mitochondrial Complex I Deficiency. Front Genet, 2017. 8: p. 113.
12. Thompson Legault, J., et al., A Metabolic Signature of Mitochondrial Dysfunction Revealed through a Monogenic Form of Leigh Syndrome. Cell Rep, 2015. 13(5): p. 981-9.
13. Cagin, U., et al., Mitochondrial retrograde signaling regulates neuronal function. Proc Natl Acad Sci USA, 2015. 112(44): p. E6000-9.
14. Merkley, E. D., et al., The succinated proteome. Mass Spectrom Rev, 2014. 33(2): p. 98-109.
15. Frizzell, N., et al., Mitochondrial stress causes increased succination of proteins in adipocytes in response to glucotoxicity. Biochem J, 2012. 445(2): p. 247-54.
16. Piroli, G. G., et al., Succination is Increased on Select Proteins in the Brainstem of the NADH dehydrogenase (ubiquinone) Fe-S protein 4 (Ndufs4) Knockout Mouse, a Model of Leigh Syndrome. Mol Cell Proteomics, 2016. 15(2): p. 445-61.
17. Chen, B., et al., Loss of Mitochondrial Ndufs4 in Striatal Medium Spiny Neurons Mediates Progressive Motor Impairment in a Mouse Model of Leigh Syndrome. Front Mol Neurosci, 2017. 10: p. 265.
18. de Haas, R., et al., Therapeutic effects of the mitochondrial ROS-redox modulator KH176 in a mammalian model of Leigh Disease. Sci Rep, 2017. 7(1): p. 11733.
19. Ferrari, M., et al., Hypoxia treatment reverses neurodegenerative disease in a mouse model of Leigh syndrome. Proc Natl Acad Sci USA, 2017. 114(21): p. E4241-E4250.
20. Jain, I. H., et al., Hypoxia as a therapy for mitochondrial disease. Science, 2016. 352(6281): p. 54-61.
21. Wang, A., et al., Rapamycin enhances survival in a Drosophila model of mitochondrial disease. Oncotarget, 2016. 7(49): p. 80131-80139.
22. Kanabus, M., et al., The pleiotropic effects of decanoic acid treatment on mitochondrial function in fibroblasts from patients with complex I deficient Leigh syndrome. J Inherit Metab Dis, 2016. 39(3): p. 415-26.
23. Martin, I., et al., A relativity concept in mesenchymal stromal cell manufacturing. Cytotherapy, 2016. 18(5): p. 613-20.
24. Uder, C., et al., Mammalian MSC from selected species: Features and applications. Cytometry A, 2017.
25. Lechanteur, C., et al., Clinical-scale expansion of mesenchymal stromal cells: a large banking experience. J Transl Med, 2016. 14(1): p. 145.
26. Yi, T., et al., Manufacture of Clinical-Grade Human Clonal Mesenchymal Stem Cell Products from Single Colony Forming Unit-Derived Colonies Based on the Subfractionation Culturing Method. Tissue Eng Part C Methods, 2015. 21(12): p. 1251-62.
27. Hanley, P. J., Therapeutic mesenchymal stromal cells: where we are headed. Methods Mol Biol, 2015. 1283: p. 1-11.
28. Nold, P., et al., Good manufacturing practice-compliant animal-free expansion of human bone marrow derived mesenchymal stroma cells in a closed hollow-fiber-based bioreactor. Biochem Biophys Res Commun, 2013. 430(1): p. 325-30.
29. Gastens, M. H., et al., Good manufacturing practice-compliant expansion of marrow-derived stem and progenitor cells for cell therapy. Cell Transplant, 2007. 16(7): p. 685-96.
30. Nicoletti, G. F., et al., Methods and procedures in adipose stem cells: state of the art and perspective for translation medicine. J Cell Physiol, 2015. 230(3): p. 489-95.
31. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.
32. Gubar, O. S., et al., Postnatal extra-embryonic tissues as a source of multiple cell types for regenerative medicine applications. Exp Oncol, 2017. 39(3): p. 186-190.
33. Lim, R., et al., A Pilot Study Evaluating the Safety of Intravenously Administered Human Amnion Epithelial Cells for the Treatment of Hepatic Fibrosis. Front Pharmacol, 2017. 8: p. 549.
34. Zlatska, A. V., et al., Endometrial stromal cells: isolation, expansion, morphological and functional properties. Exp Oncol, 2017. 39(3): p. 197-202.
35. Lan, X., et al., Stromal Cell-Derived Factor-1 Mediates Cardiac Allograft Tolerance Induced by Human Endometrial Regenerative Cell-Based Therapy. Stem Cells Transl Med, 2017.
36. Xu, X., et al., Prolongation of Cardiac Allograft Survival by Endometrial Regenerative Cells: Focusing on B-Cell Responses. Stem Cells Transl Med, 2017. 6(3): p. 778-787.
37. Rodrigues, M. C., et al., Menstrual Blood-Derived Stem Cells: In Vitro and In Vivo Characterization of Functional Effects. Adv Exp Med Biol, 2016. 951: p. 111-121.
38. James, J. L., et al., Isolation and characterisation of a novel trophoblast side-population from first trimester placentae. Reproduction, 2015. 150(5): p. 449-62.
39. Wang, Z., et al., Transplantation of human villous trophoblasts preserves cardiac function in mice with acute myocardial infarction. J Cell Mol Med, 2017. 21(10): p. 2432-2440.
40. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
41. Ducret, M., et al., A standardized procedure to obtain mesenchymal stem/stromal cells from minimally manipulated dental pulp and Wharton's jelly samples. Bull Group Int Rech Sci Stomatol Odontol, 2016. 53(1): p. e37.
42. Van Pham, P., et al., Isolation and proliferation of umbilical cord tissue derived mesenchymal stem cells for clinical applications. Cell Tissue Bank, 2016. 17(2): p. 289-302.
43. Friedman, R., et al., Umbilical cord mesenchymal stem cells: adjuvants for human cell transplantation. Biol Blood Marrow Transplant, 2007. 13(12): p. 1477-86.
44. Emnett, R. J., et al., Evaluation of Tissue Homogenization to Support the Generation of GMP-Compliant Mesenchymal Stromal Cells from the Umbilical Cord. Stem Cells Int, 2016. 2016: p. 3274054.
45. Choi, Y. S., et al., Different characteristics of mesenchymal stem cells isolated from different layers of full term placenta. PLoS One, 2017. 12(2): p. e0172642.
46. Koike, C., et al., Characterization of amniotic stem cells. Cell Reprogram, 2014. 16(4): p. 298-305.
47. Kim, S. W., et al., Amniotic mesenchymal stem cells with robust chemotactic properties are effective in the treatment of a myocardial infarction model. Int J Cardiol, 2013. 168(2): p. 1062-9.
48. Walther, G., J. Gekas, and O. F. Bertrand, Amniotic stem cells for cellular cardiomyoplasty: promises and premises. Catheter Cardiovasc Interv, 2009. 73(7): p. 917-24.
49. Ullah, M., et al., iPS-derived MSCs from an expandable bank to deliver a prodrug-converting enzyme that limits growth and metastases of human breast cancers. Cell Death Discov, 2017. 3: p. 16064.
50. Moslem, M., et al., Kindlin-2 Modulates the Survival, Differentiation, and Migration of Induced Pluripotent Cell-Derived Mesenchymal Stromal Cells. Stem Cells Int, 2017. 2017: p. 7316354.
51. Luzzani, C. D. and S. G. Miriuka, Pluripotent Stem Cells as a Robust Source of Mesenchymal Stem Cells. Stem Cell Rev, 2017. 13(1): p. 68-78.
52. Lo Cicero, A., et al., A High Throughput Phenotypic Screening reveals compounds that counteract premature osteogenic differentiation of HGPS iPS-derived mesenchymal stem cells. Sci Rep, 2016. 6: p. 34798.
53. Sheyn, D., et al., Human Induced Pluripotent Stem Cells Differentiate Into Functional Mesenchymal Stem Cells and Repair Bone Defects. Stem Cells Transl Med, 2016. 5(11): p. 1447-1460.
54. Shi, S., et al., Bone formation by human postnatal bone marrow stromal stem cells is enhanced by telomerase expression. Nat Biotechnol, 2002. 20(6): p. 587-91.
55. Grau-Monge, C., et al., Marrow-isolated adult multilineage inducible cells embedded within a biologically-inspired construct promote recovery in a mouse model of peripheral vascular disease. Biomed Mater, 2017. 12(1): p. 015024.
56. Rahnemai-Azar, A., et al., Human marrow-isolated adult multilineage-inducible (MIAMI) cells protect against peripheral vascular ischemia in a mouse model. Cytotherapy, 2011. 13(2): p. 179-92.
57. Soeder, Y., et al., First-in-Human Case Study: Multipotent Adult Progenitor Cells for Immunomodulation After Liver Transplantation. Stem Cells Transl Med, 2015. 4(8): p. 899-904.
58. Boozer, S., et al., Global Characterization and Genomic Stability of Human MultiStem, A Multipotent Adult Progenitor Cell. J Stem Cells, 2009. 4(1): p. 17-28.
59. Maziarz, R. T., et al., Single and multiple dose MultiStem (multipotent adult progenitor cell) therapy prophylaxis of acute graft-versus-host disease in myeloablative allogeneic hematopoietic cell transplantation: a phase 1 trial. Biol Blood Marrow Transplant, 2015. 21(4): p. 720-8.
60. Plessers, J., et al., Clinical-Grade Human Multipotent Adult Progenitor Cells Block CD8+ Cytotoxic T Lymphocytes. Stem Cells Transl Med, 2016. 5(12): p. 1607-1619.
61. Kebriaei, P., et al., Adult human mesenchymal stem cells added to corticosteroid therapy for the treatment of acute graft-versus-host disease. Biol Blood Marrow Transplant, 2009. 15(7): p. 804-11.
62. Allison, M., Genzyme backs Osiris, despite Prochymal flop. Nat Biotechnol, 2009. 27(11): p. 966-7.
63. Hare, J. M., et al., A randomized, double-blind, placebo-controlled, dose-escalation study of intravenous adult human mesenchymal stem cells (prochymal) after acute myocardial infarction. J Am Coll Cardiol, 2009. 54(24): p. 2277-86.
64. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.
65. Patel, A. N. and J. Genovese, Potential clinical applications of adult human mesenchymal stem cell (Prochymal(R)) therapy. Stem Cells Cloning, 2011. 4: p. 61-72.
66. Mannon, P. J., Remestemcel-L: human mesenchymal stem cells as an emerging therapy for Crohn's disease. Expert Opin Biol Ther, 2011. 11(9): p. 1249-56.
67. Wang, Y., et al., Safety, tolerability, clinical, and joint structural outcomes of a single intra-articular injection of allogeneic mesenchymal precursor cells in patients following anterior cruciate ligament reconstruction: a controlled double-blind randomised trial. Arthritis Res Ther, 2017. 19(1): p. 180.
68. Kolar, M. K., et al., The neurotrophic effects of different human dental mesenchymal stem cells. Sci Rep, 2017. 7(1): p. 12605.
69. Prather, W. R., A. Toren, and M. Meiron, Placental-derived and expanded mesenchymal stromal cells (PLX-I) to enhance the engraftment of hematopoietic stem cells derived from umbilical cord blood. Expert Opin Biol Ther, 2008. 8(8): p. 1241-50.
70. Patel, A. N., et al., Ixmyelocel-T for patients with ischaemic heart failure: a prospective randomised double-blind trial. Lancet, 2016. 387(10036): p. 2412-21.
71. Perets, N., et al., Long term beneficial effect of neurotrophic factors-secreting mesenchymal stem cells transplantation in the BTBR mouse model of autism. Behav Brain Res, 2017. 331: p. 254-260.
72. Gupta, P. K., et al., Administration of Adult Human Bone Marrow-Derived, Cultured, Pooled, Allogeneic Mesenchymal Stromal Cells in Critical Limb Ischemia Due to Buerger's Disease: Phase II Study Report Suggests Clinical Efficacy. Stem Cells Transl Med, 2017. 6(3): p. 689-699.
73. Thej, C., et al., Development of a surrogate potency assay to determine the angiogenic activity of Stempeucel(R), a pooled, ex-vivo expanded, allogeneic human bone marrow mesenchymal stromal cell product. Stem Cell Res Ther, 2017. 8(1): p. 47.
74. Zhu, M., et al., Manual isolation of adipose-derived stem cells from human lipoaspirates. J Vis Exp, 2013(79): p. e50585.
75. Van Pham, P., et al., Isolation and proliferation of umbilical cord tissue derived mesenchymal stem cells for clinical applications. Cell Tissue Bank, 2015.
76. Fazzina, R., et al., A new standardized clinical-grade protocol for banking human umbilical cord tissue cells. Transfusion, 2015. 55(12): p. 2864-73.
77. Bieback, K., Platelet lysate as replacement for fetal bovine serum in mesenchymal stromal cell cultures. Transfus Med Hemother, 2013. 40(5): p. 326-35.
78. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.
79. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
80. Can, A., F. T. Celikkan, and O. Cinar, Umbilical cord mesenchymal stromal cell transplantations: A systemic analysis of clinical trials. Cytotherapy, 2017.
81. Bilal, M., A. Haseeb, and M. A. Sher Khan, Intracoronary infusion of Wharton's jelly-derived mesenchymal stem cells: a novel treatment in patients of acute myocardial infarction. J Pak Med Assoc, 2015. 65(12): p. 1369.
82. Gao, L. R., et al., Intracoronary infusion of Wharton's jelly-derived mesenchymal stem cells in acute myocardial infarction: double-blind, randomized controlled trial. BMC Med, 2015. 13: p. 162.
83. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.
84. Liu, X., et al., A preliminary evaluation of efficacy and safety of Wharton's jelly mesenchymal stem cell transplantation in patients with type 2 diabetes mellitus. Stem Cell Res Ther, 2014. 5(2): p. 57.
85. Wu, K. H., et al., Human application of ex vivo expanded umbilical cord-derived mesenchymal stem cells: enhance hematopoiesis after cord blood transplantation. Cell Transplant, 2013. 22(11): p. 2041-51.
86. Kim, D. W., et al., Wharton's jelly-derived mesenchymal stem cells: phenotypic characterization and optimizing their therapeutic potential for clinical applications. Int J Mol Sci, 2013. 14(6): p. 11692-712.
87. Batsali, A. K., et al., Mesenchymal stem cells derived from Wharton's Jelly of the umbilical cord: biological properties and emerging clinical applications. Curr Stem Cell Res Ther, 2013. 8(2): p. 144-55.
88. Hu, J., et al., Long term effects of the implantation of Wharton's jelly-derived mesenchymal stem cells from the umbilical cord for newly-onset type 1 diabetes mellitus. Endocr J, 2013. 60(3): p. 347-57.
89. de Soure, A. M., et al., Scalable microcarrier-based manufacturing of mesenchymal stem/stromal cells. J Biotechnol, 2016. 236: p. 88-109.
90. Fernandes-Platzgummer, A., et al., Clinical-Grade Manufacturing of Therapeutic Human Mesenchymal Stem/Stromal Cells in Microcarrier-Based Culture Systems. Methods Mol Biol, 2016. 1416: p. 375-88.
91. Mizukami, A., et al., Stirred tank bioreactor culture combined with serum-/xenogeneic-free culture medium enables an efficient expansion of umbilical cord-derived mesenchymal stem/stromal cells. Biotechnol J, 2016. 11(8): p. 1048-59.
92. Smith, J. R., et al., Standardizing Umbilical Cord Mesenchymal Stromal Cells for Translation to Clinical Use: Selection of GMP-Compliant Medium and a Simplified Isolation Method. Stem Cells Int, 2016. 2016: p. 6810980.
93. Carmelo, J. G., et al., Scalable ex vivo expansion of human mesenchymal stem/stromal cells in microcarrier-based stirred culture systems. Methods Mol Biol, 2015. 1283: p. 147-59.
94. Fekete, N., et al., GMP-compliant isolation and large-scale expansion of bone marrow-derived MSC. PLoS One, 2012. 7(8): p. e43255.
95. Lange, C., et al., Accelerated and safe expansion of human mesenchymal stromal cells in animal serum free medium for transplantation and regenerative medicine. J Cell Physiol, 2007. 213(1): p. 18-26.
96. Tanthaisong, P., et al., Enhanced Chondrogenic Differentiation of Human Umbilical Cord Wharton's Jelly Derived Mesenchymal Stem Cells by GSK-3 Inhibitors. PLoS One, 2017. 12(1): p. e0168059.
97. Linares, G. R., et al., Preconditioning mesenchymal stem cells with the mood stabilizers lithium and valproic acid enhances therapeutic efficacy in a mouse model of Huntington's disease. Exp Neurol, 2016. 281: p. 81-92.
98. Ferensztajn-Rochowiak, E., et al., The effect of long-term lithium treatment of bipolar disorder on stem cells circulating in peripheral blood. World J Biol Psychiatry, 2017. 18(1): p. 54-62.
99. Ferensztajn-Rochowiak, E. and J. K. Rybakowski, The effect of lithium on hematopoietic, mesenchymal and neural stem cells. Pharmacol Rep, 2016. 68(2): p. 224-30.
100. Dong, B. T., et al., Lithium enhanced cell proliferation and differentiation of mesenchymal stem cells to neural cells in rat spinal cord. Int J Clin Exp Pathol, 2015. 8(3): p. 2473-83.
101. Zhu, Z., et al., Lithium stimulates human bone marrow derived mesenchymal stem cell proliferation through GSK-3beta-dependent beta-catenin/Wnt pathway activation. FEBS J, 2014. 281(23): p. 5371-89.
102. Tsoyi, K., et al., Carbon Monoxide Improves Efficacy of Mesenchymal Stromal Cells During Sepsis by Production of Specialized Proresolving Lipid Mediators. Crit Care Med, 2016. 44(12): p. e1236-e1245.

1. A method of treating a patient suffering from Leigh Syndrome comprising the steps of: a) selecting a patient suffering from Leigh Syndrome in need of treatment; and b) administering to said patient stem cells, and/or products derived from said stem cells at a frequency and concentration sufficient to induce a therapeutic response in said patient.

2. The method of claim 1, wherein administration of stem cells, and/or products derived from said stem cells, is performed by a route selected from the group consisting of: a) intravenous; b) intralymphatic; c) intraperitoneal; d) intrathecal; e) intraventricular; f) intra-arterial; g) subcutaneous, and h) intranasal.

3. The method of claim 1, wherein said stem cells are pluripotent stem cells.

4. The method of claim 1, wherein said pluripotent stem cells are selected from the group consisting of: a) embryonic stem cells; b) parthenogenic derived stem cells; c) inducible pluripotent stem cells; d) somatic cell nuclear transfer derived stem cells; e) cytoplasmic transfer derived stem cells; and f) stimulus-triggered acquisition of pluripotency.

5. The method of claim 1, wherein said stem cells are mesenchymal stem cells.

6. The method of claim 5, wherein said mesenchymal stem cells express a marker selected from the group consisting of: a) CD73; b) CD90; and c) CD105.

7. The method of claim 5, wherein said mesenchymal stem cells lack expression of a marker selected from the group consisting of: a) CD14; b) CD45; and c) CD34.

8. The method of claim 5, wherein said mesenchymal stem cells are derived from tissues selected from a group comprising of: a) bone marrow; b) peripheral blood; c) adipose tissue; d) mobilized peripheral blood; e) umbilical cord blood; f) Wharton's jelly; g) umbilical cord tissue; h) skeletal muscle tissue; i) subepithelial umbilical cord; j) endometrial tissue; k) menstrual blood; and l) fallopian tube tissue.

9. The method of claim 8, wherein said mesenchymal stem cells from umbilical cord tissue express markers selected from a group consisting of; a) oxidized low density lipoprotein receptor 1, b) chemokine receptor ligand 3; and c) granulocyte chemotactic protein.

10. The method of claim 8, wherein said mesenchymal stem cells from umbilical cord tissue do not express markers selected from the group consisting of: a) CD117; b) CD31; c) CD34; and CD45;

11. The method of claim 8, wherein said umbilical cord tissue mesenchymal stem cell is an isolated umbilical cord tissue cell isolated from umbilical cord tissue substantially free of blood that is capable of self-renewal and expansion in culture,

12. The method of claim 8, wherein said umbilical cord tissue derived mesenchymal stem cell expresses a marker selected from the group consisting of: a) CD10 b) CD13; c) CD44; d) CD73; e) CD90; f) PDGFr-alpha; g) PD-L2; and h) HLA-A,B,C

13. The method of claim 8, wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

14. The method of claim 8, wherein said umbilical cord tissue derived cells express markers selected from a group comprising of: a) TRA1-60; b) TRA1-81; c) SSEA3; d) SSEA4; and e) NANOG.

15. The method of claim 8, wherein said bone marrow derived mesenchymal stem cells possess markers selected from the group consisting of: a) CD73; b) CD90; and c) CD105.

16. The method of claim 8, wherein said bone marrow derived mesenchymal stem cells possess markers selected from the group consisting of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.=

17. The method of claim 1, wherein at least one lithium compound or a pharmaceutically acceptable salt thereof, is administered.

18. The method of claim 1, wherein a trait improves in the patient after treatment selected from the group consisting of: a) appetite, b) ability to walk, c) speech, and d) fine motor skills

19. The method of claim 1, wherein the administration comprises an intranasal administration followed by an intravenous administration.

20. The method of claim 1, wherein subsequent stem cells treatments are administered within 5 months from the previous treatment.
